(19) United States
(12) Patent Application Publication
(10) Pub. No.: US 2010/0113802 A1
(43) Pub. Date: May 6, 2010

(54) PROCESS FOR PREPARING AMORPHOUS ATORVASTATIN HEMI CALCIUM SALT AND ITS ITERMEDIATE

(75) Inventors: Indravadan Ambalal Modi, Ahmedabad (IN); Pratima Jain, Ahmedabad (IN); Amarsingh L. Rajput, Ahmedabad (IN); Prabhakar Motiram Tekade, Ahmedabad (IN); Atul Chhotalal Joshi, Ahmedabad (IN); Ravi Ponnaiah, Ahmedabad (IN); Bakulesh Mafatlal Khamar, Ahmedabad (IN)

Correspondence Address: DARBY & DARBY P.C., P.O. BOX 770, Church Street Station, New York, NY, 10008-0770, US

(73) Assignee: Cadila Pharmaceuticals Limited, Ahmedabad (IN)

(21) Appl. No.: 12/513,346

(22) PCT Filed: Oct. 29, 2007
(86) PCT No.: PCT/IB07/03251

§ 371 Date: Jan. 11, 2010

(30) Foreign Application Priority Data
Nov. 2, 2006 (IN) 1829/MUM/2006
Feb. 19, 2007 (IN) 334/MUM/2007

(51) Int. Cl. C07D 207/34

(52) U.S. Cl. 548/537

(57) ABSTRACT

The invention relates to the HMG-CoA reductase inhibitor in particular to Atorvastatin Hemi-calcium. The present invention is directed to novel processes for preparing amorphous form of Atorvastatin hemi calcium and their intermediate in high purity.

### CROSS-REFERENCE TO PRIOR APPLICATIONS

This is a United States national phase application under 35 U.S.C. §371 of International Patent Application No. PCT/IB2007/003251 filed Oct. 29, 2007 which claims the benefit of Indian Patent Application Nos. 1829/MUM/2006 filed Nov. 2, 2006, and 334/MUM/2007 filed Feb. 20, 2007. The International Application was published on May 8, 2008 as WO 2008/053312 under PCT Article 21(2).

### FIELD OF THE INVENTION

The general field of invention relates to the HMG-CoA reductase inhibitor in particular to Atorvastatin hemi-calcium. The present invention is more specifically relates to a novel processes for the preparation of amorphous form of Atorvastatin hemi-calcium and their intermediate in high purity.

### BACKGROUND OF THE INVENTION

Atorvastatin calcium is chemically known as [R—(R*,R*)]-2-(4-Iluorophenyl)-(3,8-dihydroxy-5-(1-methylethyl)-3-phenyl-4-[(phenylamino)carbonyl]-1H-pyrrole-1-heptanoic acid calcium salt (2:1 salt) having the structural formula (I) as follows.

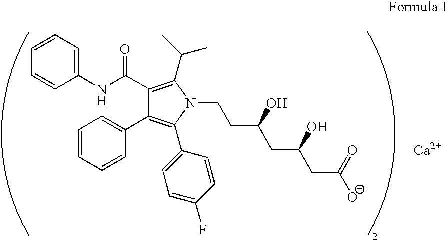

Atorvastatin is known to be therapeutically useful as an inhibitor of the enzyme 3-hydroxy-3-methylglutaryl coenzyme a reductase (HMG-CoA reductase inhibitor), and is used for the treatment of hyperlipidemia and hypercholesterolemia.

It is known that the amorphous form of Atorvastatin hemi-calcium exhibits different dissolution characteristics and bioavailability patterns compared to its crystalline forms. Atorvastatin hemi-calcium is slightly water-soluble, and it has been found that as comparison to crystalline forms, amorphous form of Atorvastatin hemi-calcium facilitates the bioavailability in the body.

U.S. Pat. No. 5,273,995 disclose the hemi calcium salt of Atorvastatin. U.S. Pat. Nos. 5,003,080; 5,103,045; 5,103,024; 5,124,482; 5,149,837; 5,155,251; 5,216,174; 5,245,047; 5,248,793; 5,280,126; 5,342,952 and 5,397,792 discloses various processes and key intermediate which may useful for preparing Atorvastatin hemi-calcium salt. The processes resulting Atorvastatin hemi-calcium in the above-mentioned US patents does not give amorphous form consistently but gives a mixture of crystalline and amorphous forms and are not suitable for large-scale production.

WO02/057228 describes the preparation of amorphous Atorvastatin hemi-calcium and its hydrates which comprises dissolving heterogeneous mixture of Atorvastatin hemi-calcium in a non-hydroxylic solvent; followed by adding a non-hydroxylic solvent or adding the dissolved Atorvastatin to the non-hydroxylic solvent to precipitate out Atorvastatin hemi-calcium; and finally solvent was removed by the filtration to afford amorphous Atorvastatin hemi-calcium WO02/083637 describes preparation of Atorvastatin hemi-calcium in amorphous form comprising the treatment of diol protected tert-butyl ester with a methanolic solution in the presence of an aqueous acid; followed by adding aqueous hydroxide solution to the reaction mixture. The resulting compound was treated with calcium chloride to give crude amorphous Atorvastatin hemi-calcium salt. The crude was treated with excess volume of methanol and precipitation in afforded by adding methanolic solution of Atorvastatin hemi-calcium in to water. WO02/083638 describes substantially similar invention wherein the crude product is isolated with activated carbon in aqueous ethyl acetate to recover the product by addition of non polar hydrocarbon solvent filtration. Upon drying the produce produces amorphous Atorvastatin hemi-calcium.

U.S. Pat. No. 6,528,660 (WO00/71116) describes a process for the preparation of amorphous Atorvastatin hemi-calcium and hydrates which comprises dissolving crystalline Atorvastatin hemi-calcium in a non-hydroxylic solvent; followed by adding a non-polar hydrocarbon anti-solvent or adding the dissolved Atorvastatin to the non-polar anti-solvent to precipitate out Atorvastatin hemi-calcium; and removing the solvent by filtration to afford amorphous Atorvastatin hemi-calcium. U.S. Pat. No. 6,613,916 (WO01/042209) describes a process for the preparation of Atorvastatin in an amorphous form by precipitating the Atorvastatin using a solvent of a second type from a solution of Atorvastatin which is provided with a solvent of a first type. This process is useful for the conversion of Atorvastatin in a crystalline form into Atorvastatin in an amorphous form. U.S. Pat. No. 646,133 (WO01/028999) describes a process for the preparation of amorphous Atorvastatin hemi-calcium by recrystallization of crude Atorvastatin from an organic solvent which comprises dissolving crude amorphous Atorvastatin hemi-calcium in a lower alkanol containing 2-4 carbon atoms or a mixture of such alkanols under heating and isolating the amorphous Atorvastatin hemi-calcium precipitated after cooling.

WO03/093233 describes a process for the preparation of Atorvastatin hemi-calcium salt in amorphous form comprising: a) dissolving the Atorvastatin hemi-calcium salt in an organic solvent miscible with water, b) gradually adding said solution to water while stirring, c) filtering and vacuum drying the solid obtained. WO03/099785 describes a process for the preparation of amorphous Atorvastatin hemi-calcium. In essence, the process comprises dissolving form—I or a mixture of crystalline and amorphous Atorvastatin hemi-calcium in a solvent consisting of an aliphatic acyclic ketone, filtering the solution and removing the solvent at 40 to 50° C. under vacuum.

U.S. Pat. No. 6,750,353 (WO02/059087) discloses that Atorvastatin hemi-calcium can exist in an amorphous form or in one of the crystalline forms (Form I, Form II, Form III and Form IV).

WO2004/085391 describes a process for the synthesis of amorphous Atorvastatin hemi-calcium, which consists of dissolving the salt of the formula (I) of Atorvastatin acid formed with a basic amino acid (I); in a mixture of water and a water miscible organic solvent, adding an aqueous solution of a water soluble calcium salt to the solution and isolating the amorphous Atorvastatin hemi-calcium having high purity by filtration. WO2004/089895 describes the process of preparing amorphous Atorvastatin hemi-calcium without intermediate isolation of crystal or undefined mixture of crystal and amorphous Atorvastatin hemi-calcium, respectively. The formation of Atorvastatin hemi-calcium salt is carried out in a mixture of chlorinated organic solvent or cyclic hydrocarbon solvent, respectively, the non-hydroxylic organic solvent, and water, the source of calcium ions is calcium acetate or calcium chloride, respectively. US2004/0242670 describes a process for the preparation of amorphous form of Atorvastatin hemi-calcium, which comprises the conversion of the crystalline Atorvastatin hemi-calcium to amorphous Atorvastatin hemi-calcium. WO2005/005384 describes a process for the preparation of amorphous Atorvastatin hemi-calcium salt (2:1) from Atorvastatin tert-butyl ester comprising: (a) dissolving Atorvastatin tert-butyl ester in a solvent, (b) adding an aqueous alkaline or alkaline earth metal hydroxide solution, (c) removing of the solvent, b) adding water and a water non soluble solvent, e) adding an aqueous calcium salt solution, 1) separation of the phases and removing of the solvent to obtain desired amorphous Atorvastatin hemi-calcium and hydrates thereof. The process disclosed herein gives amorphous form directly without interconversion of any crystalline form into amorphous form.

US2005/0032880 (WO2004/110407) describes formation of amorphous Atorvastatin comprising the steps of dissolving Atorvastatin in a hydroxylic solvent, followed by rapidly evaporating the solvent. U.S. Pat. No. 6,891,047 describes a process for preparing Atorvastatin in an amorphous form by precipitating the Atorvastatin using a solvent of a second type from a solution of Atorvastatin which is provided with a solvent of a first type. This process is useful for the conversion of Atorvastatin in a crystalline form into Atorvastatin in an amorphous form. The solvent of the first type is a chlorinated solvent selected from the group consisting of chloroform, methylene chloride, a polar solvent selected from the group consisting of DMF, DMSO or a mixture thereof and the solvent of the second type comprises at least one solvent selected from the group consisting of ether solvents and aliphatic solvents. US2005/0119493 (WO2003/018547) describes preparation of amorphous Atorvastatin hemi-calcium salt (2:1) comprising hydrolyzing the lactone form of Atorvastatin with aqueous alkali or alkaline earth metal base, extracting with organic solvent the reaction mixture and adding the same to an anti-solvent to precipitate the product and finally filtering the product to afford amorphous Atorvastatin hemi-calcium. The process also comprises the preparation of amorphous Atorvastatin hemi-calcium salt (2:1) from its crystalline form.

US2005/0131055 (WO03/068739) describes a method of manufacturing an amorphous form of the hemi-calcium salt of (3R,5R) 7-[3-phenyl-4-phenylcarbamoyl-2-(4-fluorophenyl)-5-isopropyl-pyrrol-1-yl]-3,5-dihydroxyheptanoic acid of formula (I), in which (3R,5R) 7-[3-phenyl-4-phenylcarbamoyl-2-(4-fluorophenyl)-5-isopropyl-pyrrol-1-yl]-3,5-dihydroxy heptanoic acid or its salt with a cation M+ wherein M+ is either a cation of an alkali metal or an ammonium cation of formula RnN(+)H(4-n) wherein R is lower C₁-C₅ alkyl, n may reach values ranging between 0 and 3 is, without isolating the intermediate in the form of the hemi-calcium salt or of another salt, acid or lactone, converted, in a solution, by the treatment with the calcium salt or calcium hydroxide, or a calcium C₁-C₅ alcoholate. US2005/0165242 describes a process for the preparation of amorphous Atorvastatin hemi-calcium and hydrates thereof, which comprises: (a) hydrolysis of the precursor lactone using sodium hydroxide to form Atorvastatin sodium salt solution; (b) addition of the Atorvastatin sodium salt solution to a calcium chloride or calcium acetate solution in the absence or presence of seeds of amorphous Atorvastatin hemi-calcium; and (c) isolation of the resultant amorphous Atorvastatin hemi-calcium salt by filtration and drying. WO2005/073187 describes a process for the preparation of amorphous Atorvastatin hemi-calcium which comprising: (a) hydrolysis of the Atorvastatin lactone of formula II to form Atorvastatin sodium salt solution; (b) addition of the Atorvastatin sodium salt solution to an aqueous calcium chloride or calcium acetate solution; (c) isolation of the product; and (d) drying to afford amorphous Atorvastatin hemi-calcium salt.

WO2005/092852 describes a process for the production of amorphous Atorvastatin hemi-calcium and stabilized amorphous Atorvastatin hemi-calcium. WO2005/100313 describes preparation of amorphous Atorvastatin hemi-calcium comprises the steps of dissolving Atorvastatin hemi-calcium in a solvent to form a solution, followed by adding the solution to a mixture comprising a non-solvent and a hydroxylic solvent to afford amorphous Atorvastatin. US2005/0261360, US2005/0261359, US2005/0267198 describes novel forms of Atorvastatin designated Forms VI, VIII, IX, X, XI and XII and novel processes for their preparation as well as processes for preparing Atorvastatin Forms I, II, IV, V and amorphous Atorvastatin. WO2006/011155 describes one pot process for the preparation of amorphous Atorvastatin hemi-calcium by treating solution of ATV-1 in a water miscible polar organic solvent with an aqueous acid, neutralizing and hydrolyzing at a temperature ranging between 40° C. to 55° C. using aqueous alkali hydroxide, removing the polar solvent under vacuum to reduce the volume to one fourth, adding water, methanol and methyl-t-butylether, stirring, separating the aqueous layer, extracting further aqueous layer with ethyl acetate-n-hexane mixture, collecting aqueous layer after extraction, adjusting the pH between 7.5 to 8.5, stirring at a temperature between 40°-55° C., adding aqueous calcium acetate solution in portions, seeding with amorphous Atorvastatin hemi-calcium.

WO2006/021969 describes a process of the preparation of amorphous Atorvastatin hemi-calcium starting from (6-{2-[2-(4-fluorophenyl)-5-isopropyl-3-phenyl-4-phenylcarbamoyl-pyrrolidin-1-yl]-ethyl}-2-phenyl-[1,3,2]-dioxaborinan-4-yl)-acetic acid tert butyl ester. WO2006/039441 describes amorphous Atorvastatin hemi-calcium having an enhanced stability contains about 2 to about 8 percent by weight water. A process for preparing the amorphous Atorvastatin hemi-calcium and a packaging system for maintaining the stability are described. WO2006/046109 describes A process for forming amorphous Atorvastatin comprising the steps of dissolving Atorvastatin in a non-hydroxylic solvent and removing the solvent by freeze-drying, as well as processes of dissolving Atorvastatin in a hydroxylic solvent with a solubilizing agent or an alkalizing agent or an antioxidant and removing the solvent by freeze-drying to afford amorphous Atorvastatin. WO2006/048888 describes a process for the preparation of amorphous Atorvastatin hemi-calcium comprising: (i) dissolving Atorvastatin hemi-calcium in an organic solvent selected from the group comprising 1,4-dioxane, acetonitrile, toluene, anisole and tert. butanol or mixtures; (ii) adding anti-solvent to the solution prepared in (i) selected from the group comprising cyclohexane, n-hexane, n-heptane, methyl tert. butyl ether and methanol or a mixture (iii) separating the resulting precipitate to obtain the amorphous Atorvastatin hemi-calcium.

US2006/0106230 (WO2006/045018) describe processes for the preparation of amorphous Atorvastatin hemi-calcium salt which involve dissolving Atorvastatin hemi-calcium salt in certain organic solvents, and removing the solvent such as by spray drying, rapid vacuum evaporation, and/or thin film evaporation. Preferred embodiments of these processes for preparing amorphous Atorvastatin hemi-calcium salt are reproducible, applicable on a large scale, and do not involve the use of hydrocarbons. US2006/0128971 describes a Process for preparing Atorvastatin hemi-calcium salt in amorphous form comprising: a) dissolving the Atorvastatin hemi-calcium salt in an organic solvent miscible with water, b) gradually adding said solution to water while stirring, c) filtering and vacuum drying the solid obtained. U.S. Pat. No. 6,087,511 (WO97/03960, U.S. Pat. No. 6,274,740) describes preparation of amorphous Atorvastatin by dissolving crystalline Form I Atorvastatin in a non-hydroxylic solvent followed by removal of the solvent.

The prior art processes of amorphous Atorvastatin are not appropriate for commercial production. There is a need to provide processes for the preparation of amorphous Atorvastatin without the co-formation of crystalline forms. It is known that certain protected 3,5-dihydroxy heptanoic derivatives are important intermediates for the synthesis of Atorvastatin, which is an inhibitor of the 3-hydroxy-3-methyl glutaryl coenzyme-A [HMG-CoA]. U.S. Pat. No. 5,216,174 describes generally that the Paal Knorr reaction can be performed on an acetonide-protected 7-amino-3,5-dihydroxy heptanoic acid tert-butyl ester with 1,4-diketone in an inert solvent or solvents such as for example, hexane, toluene at about reflux temperature of the solvent and that the product is not isolated but is treated directly with acid to remove the acetonide protecting group. Further, K. L. Baumann et al. describes in tetrahedron Lett. 1992, 33, 2283-84 the preparation of dimethyl ketol of the Atorvastatin tertiary butyl ester (II) by a Paal-Knorr pyrrole synthesis using a ternary solvent mixture of toluene-heptane-tetrahydrofuran (1:4:1) with catalysis by pivalic acid and conversion of (I) to Atorvastatin hemi-calcium without isolating any intermediates.

U.S. Pat. No. 5,298,627 describes a process for preparing Atorvastatin wherein the reaction of amine with 1,4-diketone is carried out in Heptane:THF:Toluene, in volume ratio [2:1:1], in the presence of pivalic acid as catalyst. The product was an acetonide protected 3,5-dihydroxy-7-pyrrol-1-yl heptanoic acid amide. After cleaving the acetonide, the amide group was hydrolyzed to the carboxylic acid with sodium hydroxide to give Atorvastatin as the sodium salt. U.S. Pat. No. 5,397,792 describes condensation between a diketone and an amine wherein the condensation is carried out in Heptane:THF:Toluene in volume ratio [6:10:5] in the presence of pivalic acid as catalyst. WO1/72706 describes the Paal-Knorr reaction in non-polar solvent like xylene and acetonitrile.

WO 2004/046105 describes the Paal Knorr reaction of a compound of formula-(II) with a compound of formula-(III) using pivalic acid in THF under reflux and with evaporative removal of water.

Solvents used in Paal-Knorr reaction are hexane, heptane, cyclohexane, xylene, MTBE, diisopropyl ether, acetonitrile, toluene, THF. Preferred solvents are mixtures of heptane, THF, and toluene. Suitable acid catalysts are acetic acid, butyric acid, pivalic acid, benzoic acid and trichloro acetic acid phenols and cresols.

### SUMMARY OF THE INVENTION

The process for the preparation of an amorphous form of Atorvastatin hemi-calcium salt comprises the step of reacting a solution (βR,δR)2-(4-fluorophenyl)-β,δ-dihydroxy-5-(1-methylethyl)-3-phenyl-4-[(phenylamino)carbonyl]-1H-Pyrrole-1-heptanoic acid t-butyl ester (formula II) in an organic solvent, with aqueous alkali base to give an alkali metal salt of Atorvastatin which on reaction with a calcium source followed by extraction of Atorvastatin hemi-calcium using an anti-solvent results in an amorphous form.

In a preferred embodiment, the objective of present invention is to provide an improved and commercially feasible process for the preparation of amorphous Atorvastatin hemi calcium salt. Another object of the invention is to provide an appropriate solvent system for the production of amorphous Atorvastatin hemi calcium without the co-production of other crystalline form. Yet another objective of the present invention is to provide alternative reagents as source of calcium ions for the preparation of the amorphous Atorvastatin hemi calcium salt.

The present invention provides a commercially feasible process for the preparation of [R—(R*,R*)]-2-(4-fluorophenyl)-β,δ-dioxane-5-(1-methylethyl)-3-phenyl-4-[(phenylamino)carbonyl]-1H-pyrrole-1-heptanoic acid tert-butyl ester, compound of formula III (Pyrrol intermediate). Aspect of the present invention is to get “pyrrol intermediate” in higher yield and purity. The preferred objective is also to obtain the “pyrrol intermediate” by using single solvent, which is therefore easy to recover and recycle. The objective is to obtain the “pyrrol intermediate” with reduced byproducts, thereby resulting Atorvastatin hemi-calcium in higher purity.

### BRIEF DESCRIPTION OF THE DRAWINGS

FIG. 1 shows a powder X-ray diffraction pattern of amorphous Atorvastatin Hemi-calcium

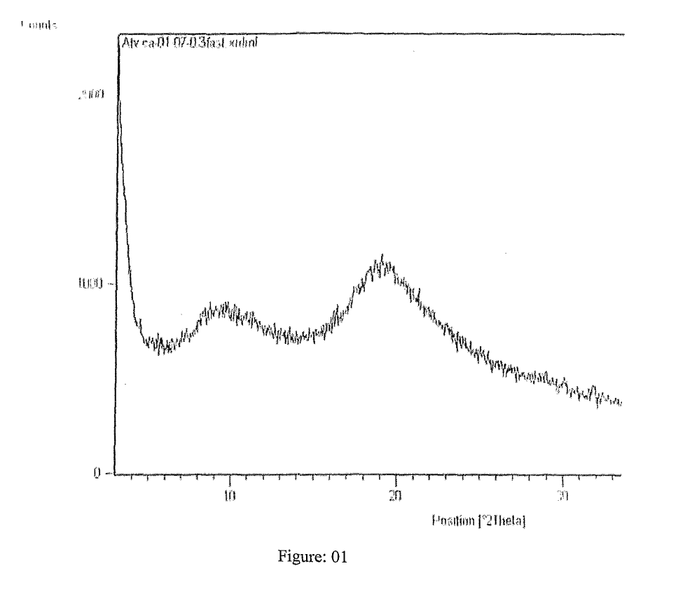

### DETAILED DESCRIPTION OF THE INVENTION

In accordance with the present invention, amorphous Atorvastatin hemi calcium is prepared by a process comprising steps of preparing the solution of (βR,δR)2-(4-fluorophenyl)-β,δ-dihydroxy-5-(1-methylethyl)-3-phenyl-4-[(phenylamino)carbonyl]-1H-Pyrrole-1-heptanoic acid t-butyl ester (formula II) in acetonitrile followed by saponifying the ester with alkali to give Atorvastatin alkali salt. The alkali salt of Atorvastatin on reaction with calcium source gives Atorvastatin hemi-calcium which is extracted with 2-methylTHF. The preferable alkali is an NaOH, KOH. More preferably the suitable alkali is NaOH. The Atorvastatin hemi-calcium is precipitated by adding the Atorvastatin hemi-calcium salt solution to an anti solvent selected from cyclohexane, n-hexane, n-heptane, methyl t-butyl ether or a mixture or by adding an anti-solvent to the solution of Atorvastatin hemi-calcium. The resulting product is in amorphous form.

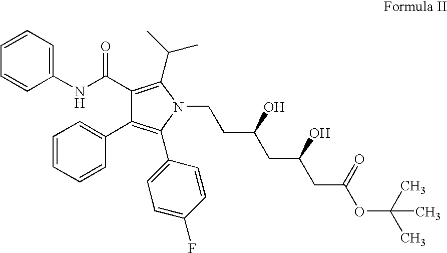

It has surprisingly been found that amorphous Atorvastatin hemi calcium can be formed by adding a Atorvastatin hemi-calcium salt solution (in 2-methylTHF) to an anti solvent selected from cyclohexane, n-hexane, n-heptane, methyl t-butyl ether or a mixture or adding anti-solvent to 2-methylTHF solution of Atorvastatin hemi-calcium,

The solvent used for the formation and extraction of amorphous Atorvastatin hemi-calcium is having an added advantage over prior disclosed organic solvent or mixture thereof. The process for the preparation of the amorphous Atorvastatin hemi calcium using a compound of formula (II), wherein the process comprises dissolving compound (II) an organic solvent wherein the preferred organic solvent is acetonitrile at room temperature, adding aqueous alkali preferably, sodium hydroxide solution with stirring at 25-100° C., preferably at 45-50° C. followed by addition calcium source. The preferred addition of calcium source is a solution of calcium gluconate or calcium acetate with stirring at 45-50° C. Atorvastatin hemi-calcium is extracted with 2-methyl THF and organic phase is separated. The filtered solution of formula II is concentrated and added to a mixture of n-hexane and methyl t-butyl ether, followed by separation of the precipitate and drying under vacuum. The compound of formula (II) involved in above process is prepared by procedure as mentioned in prior art literature.

Another embodiment of the present invention relates to an improved process for the preparation of highly pure chiral enantiomers [R—(R*,R*)]-2-(4-fluorophenyl)-β,δ-dioxane-5-(1-methylethyl)-3-phenyl-4-[(phenylamino)carbonyl]-1H-pyrrole-1-heptanoic acid tert-butyl ester, compound of formula III which is useful as an intermediate for the preparation of Atorvastatin hemi-calcium.

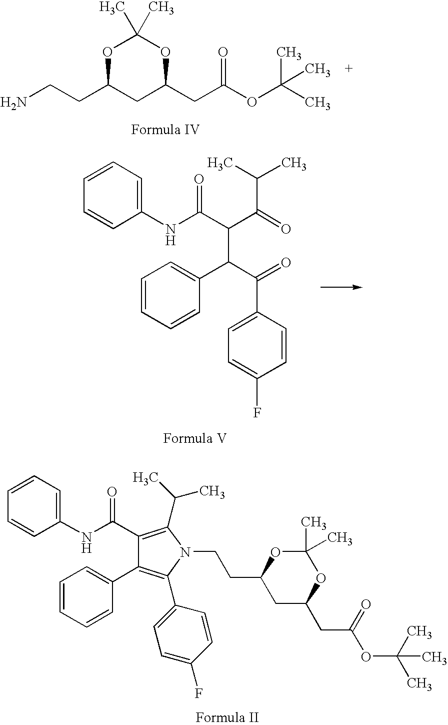

The reaction of 1,4-dicarbonyl compound in the presence of an ammonia or primary amine to give substituted pyrrole which is known as Paal-Knorr reaction. The said reaction is promoted by heating, removal of water using an acid catalyst.

In accordance with the present invention, compound of formula II is prepared via the reaction consisting steps of:
(a) reaction of (4R-6R)-1′,1′-dimethylethyl-6′-aminoethyl-2,2-dimethyl-1,3-dioxane-4-acetate (compound of formula IV) with 2-[2-(4-Fluoro-phenyl)-2-oxo-1-phenyl-ethyl]-4-methyl-3-oxo-pentanoic acid phenylamide (compound of formula V), using an acid catalyst, preferably pivalic acid in 2-Methyl tetrahydrofuran by azeotropically removal of water;
(b) concentrating the reaction mass by partial removal of solvent followed by separating the product or isolating the product by complete removal of solvent followed by purification from lower alcohol or aqueous alcohol. The preferred lower alcohol is C₁-C₄ alcohol or mixtures thereof.

In a preferred embodiment, the Paal-Knorr reaction is carried out in a single solvent with pivalic acid as an acid catalyst. The preferred solvent is 2-Methyl THF. After dissolving the reactants and catalyst in 2-Methyl THF, the solution is heated to reflux, with continuous separation of water by Dean Stark, till no further water of reaction is separated. The reaction mixture is then concentrated, either on a rotary evaporator or by distillation. The product is isolated from concentrated solution by cooling followed by filtration. Optionally, the residue obtained by the evaporation of solvent is dissolved in lower alkyl alcohol such as methanol, ethanol or isopropanol and the product is isolated by crystallization or by precipitation by addition of water to obtain the “pyrrole intermediate” with purity above 99%. The use of 2-methyl THF as solvent during preparation of “pyrrole intermediate” reduces the amount of side products formation which is resulting in improved quality of “pyrrole intermediate”. The pure pyrrole intermediate may prefer to convert into highly pure Atorvastatin hemi-calcium in amorphous form. The solvent system may preferably recycled.

The invention is now illustrated with some non-limiting examples.

### Example-1

### Preparation of [R—(R*,R*)]-2-(4-fluorophenyl)-β,δ-dioxane-5-(1-methylethyl)-3-phenyl-4-[(phenylamino)carbonyl]-1H-pyrrole-1-heptanoic acid tert-butyl ester, compound of formula III

(4R-6R)-6-aminoethyl-2,2-dimethyl-1,3-dioxane-4-acetic acid tert-butyl ester (compound of formula IV) (50 gms); 2-[2-(4-Fluoro-phenyl)-2-oxo-1-phenyl-ethyl]-4-methyl-3-oxo-pentanoic acid phenylamide (compound of formula V) (68.9 gm); pivalic acid (11.96 gm) and 2-Methyl THF (750 ml) were stirred with reflux, water is removed through dean stark apparatus during the course of reaction. The mixture was refluxed for about 30-35 hours. After cooling, the reaction mixture was concentrated. Thus obtained oily residue is dissolved in 2-propanol (350 ml) with heating. The mixture cooled slowly to room temperature and stirred for 2 hours, further cooled to 15-20° C. and stirred for one hour. The solid precipitate out which is filtered, washed with IPA and dried at 60° C. overnight to give the title compound as off while solid (63 gm; Purity: >99%).

### Example-2

### Preparation of [R—(R*,R*)]-2-(4-fluorophenyl)-β,δ-dioxane-5-(1-methylethyl)-3-phenyl-4-[(phenylamino)carbonyl]-1-1H-pyrrole-1-heptanoic acid tert-butyl ester, compound of formula III

(4R-6R)-6-aminoethyl-2,2-dimethyl-1,3-dioxane-4-acetic acid tert-butyl ester (compound of formula IV) (50 gms); 2-[2-(4-Fluoro-phenyl)-2-oxo-1-phenyl-ethyl]-4-methyl-3-oxo-pentanoic acid phenylamide (compound of formula V) (68.9 gm); pivalic acid (11.96 gm) and 2-Methyl THF (750 ml) were stirred with reflux, water is removed through dean stark apparatus during the course of reaction. Thus obtained oily residue is dissolved in 2-propanol (350 ml) with heating. Water (138 ml) was added drop wise. The reaction mixture was cooled slowly till reaches room temperature and stirred for 2 hours. The solid precipitate out which is filtered, washed with 2-propanol and dried overnight at 60° C. to give [R—(R*,R*)]-2-(4-fluorophenyl)-β,δ-dioxane-5-(1-methylethyl)-3-phenyl-4-[(phenylamino)carbonyl]-1H-pyrrole-1-heptanoic acid tert-butyl ester as off white solid, (63 gm; Purity: >99%).

### Example-3

### Preparation of [R—(R*,R*)]-2-(4-fluorophenyl)-β,δ-dioxane-5-(1-methylethyl)-3-phenyl-4-[(phenylamino)carbonyl]-1-1H-pyrrole-1-heptanoic acid tert-butyl ester, compound of formula III

(4R-6R)-6-aminoethyl-2,2-dimethyl-1,3-dioxane-4-acetic acid tert-butyl ester (compound of formula IV) (50 gms); 2-[2-(4-Fluoro-phenyl)-2-oxo-1-phenyl-ethyl]-4-methyl-3-oxo-pentanoic acid phenylamide (compound of formula V) (68.9 gm); pivalic acid (11.96 gm) and 2-Methyl THF (750 ml) were stirred and refulx. The water is removed during the course of reaction. After reaction completion, about (400 ml) of solvent was distilled out and then cooled to 25-30° C., stirred for 2 hours further cooled to 10-15° C., Stirred for 30 minutes and Filtered, washed with 2-methyl THF (100 ml), and dried at 55-60° C. overnight to give the title compound as an off white solid. (60 gm; Purity: >99%).

### Example 4

### Preparation of Amorphous Atorvastatin Hemi-Calcium (Formula I)

(βR,δR)2-(4-fluorophenyl)-β,δ-dihydroxy-5-(1-methylethyl)-3-phenyl-4-[(phenyl amino)carbonyl]-1H-Pyrrole-1-heptanoic acid t-butyl ester (formula II) (50 gm) was dissolved in acetonitrile (100 ml). Sodium hydroxide solution (3.58 gm/350 ml water) was added with stirring and the reaction mass was stirred at 45-50° C. for 5-6 hrs. After the completion of the reaction, calcium acetate solution (7.73 gm/400 ml water) was added with stirring, the reaction mass was stirred for one hour at 45-50° C. 2-Methyltetrahydrofurane [2-Methyl THF] (500 ml) was added and stirred for 10 minutes. The layers were separated. The aqueous layer was extracted with 2-Methyltetrahydrofurane (250 ml) and stirred for 10 minutes. The layers were separated. The organic layers were combined and the unwanted material were removed by filtration. The filtrate was then concentrated up to 150 ml and added to n-hexane: MTBE (600 ml:600 ml). The precipitated material was stirred at room temperature, filtered and dried to give amorphous Atorvastatin hemi-calcium.

### Wt.=41 gm

### Example 5

### Preparation of Amorphous Atorvastatin Hemi-Calcium (Formula I)

(βR,δR)2-(4-fluorophenyl)-β,δ-dihydroxy-5-(1-methylethyl)-3-phenyl-4-[(phenyl amino)carbonyl]-1H-Pyrrole-1-heptanoic acid t-butyl ester (formula II) (50 gm) was dissolved in acetonitrile (100 ml). Sodium hydroxide solution (3.58 gm/350 ml water) was added with stirring and the reaction mass was stirred at 45-50° C. for 5-6 hrs. After the completion of the reaction, calcium acetate solution (7.73 gm/400 ml water) was added with stirring, the reaction mass was stirred for one hour at 45-50° C. 2-Methyltetrahydrofurane (500 ml) was added and stirred for 10 minutes. The layers were separated. The aqueous layer was extracted with 2-Methyltetrahydrofurane (250 ml) and stirred for 10 minutes. The layers were separated. The organic layers were combined; the undissolved material was removed by filtration. The filtrate was then concentrated up to 150 ml, to which a mixture of n-hexane: MTBE (600 ml:600 ml) was added. The precipitated material was stirred at room temperature, filtered and dried to give amorphous Atorvastatin hemi-calcium.

### Wt.=40 gm

### Example 6

### Preparation of Amorphous Atorvastatin Hemi-Calcium (Formula I)

(βR,δR)2-(4-fluorophenyl)-β,δ-dihydroxy-5-(1-methylethyl)-3-phenyl-4-[(phenyl amino)carbonyl]-1H-Pyrrole-1-heptanoic acid t-butyl ester (formula II) (50 gm) was dissolved in acetonitrile (100 ml). Sodium hydroxide solution (3.58 gm/350 ml water) was added with stirring and the reaction mass was stirred at 45-50° C. for 5-6 hrs. After the completion of the reaction, calcium acetate solution (7.73 gm/400 ml water) was added with stirring, the reaction mass was stirred for one hour at 45-50° C. 2-Methyltetrahydrofurane (500 ml) was added and stirred for 10 minutes. The layers were separated. The aqueous layer was extracted with 2-Methyltetrahydrofurane (250 ml) and stirred for 10 minutes. The layers were separated. The organic layers were combined and the undissolved material was removed by filtration. The filtrate was concentrated up to 150 ml and added to n-heptane (1200 ml). The precipitated material was stirred at room temperature, filtered and dried to give amorphous Atorvastatin hemi-calcium.

### Wt.=42 gm

### Example 7

### Preparation of Amorphous Atorvastatin Hemi-Calcium (Formula I)

(βR,δR)2-(4-fluorophenyl)-β,δ-dihydroxy-5-(1-methylethyl)-3-phenyl-4-[(phenyl amino)carbonyl]-1H-Pyrrole-1-heptanoic acid t-butyl ester (formula II) (50 gm) was dissolved in acetonitrile (100 ml). Sodium hydroxide solution (3.58 gm/350 ml water) was added with stirring and the reaction mass was stirred at 45-50° C. for 5-6 hrs. After the completion of the reaction, calcium gluconate solution (22.4 gm/400 ml water) was added with stirring, the reaction mass was stirred for one hour at 45-50° C. 2-Methyltetrahydrofurane [2-Methyl THF] (500 ml) was added and stirred for 10 minutes. The layers were separated. The aqueous layer was extracted with 2-Methyltetrahydrofurane (250 ml) and stirred for 10 minutes. The layers were separated. The organic layers were combined and the undissolved material was removed by filtration. The filtrate was then concentrated up to 150 ml and added to n-hexane: MTBE (600 ml:600 ml). The precipitated material was stirred at room temperature, filtered and dried to give amorphous Atorvastatin hemi-calcium.

### Wt.=41 gm

### Example 8

### Preparation of Amorphous Atorvastatin Hemi-Calcium (Formula I)

Atorvastatin hemi-calcium (5 gm) was dissolved in 2-methylTHF (20 ml), filter the traces. The solution obtained was slowly added to n-hexane: methyl t-butyl ether mixture (60 ml:60 ml) to give precipitate. The product was filtered and dried under vacuum.

Wt.=4 gm

1. A process for the preparation of an amorphous form of Atorvastatin hemi-calcium salt comprising:
a) reaction of (βR,δR)2-(4-fluorophenyl)-β,δ-dihydroxy-5-(1-methylethyl)-3-phenyl-4-[(phenylamino)carbonyl]-1H-Pyrrole-1-heptanoic acid t-butyl ester having structural formula II, 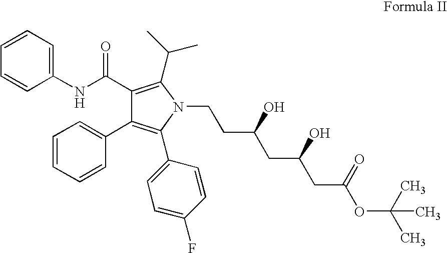
with an aqueous alkali in an organic solvent,
b) reacting the alkali metal salt of Atorvastatin with a calcium source,
c) extracting the Atorvastatin calcium with 2-methylTHF, and
d) treating the resulting solution with an anti-solvent to give amorphous Atorvastatin calcium.

2. The process of claim 1, wherein the organic solvent in step 1(a) is acetonitrile.

3. The process of claim 1, wherein the aqueous alkali in step 1(a) is selected from aqueous NaOH or KOH.

4. The process of claim 1, wherein the aqueous alkali is aqueous NaOH.

5. The process of claim 1, wherein the calcium source in step 1(b) calcium gluconate or calcium acetate.

6. The process of claim 1 wherein, the extraction of Atorvastatin calcium involves
(i) dissolving the reaction mixture in 2-methyltetrahydrofuran, and
(ii) (A) separating and concentrating the organic layer followed by addition of an anti-solvent to the solution, or
(B) adding Atorvastatin calcium solution in 2-methyltetrahydrofuran to an anti-solvent,
to provide Atorvastatin hemi-calcium in an amorphous form.

7. A process for the preparation of Atorvastatin hemi calcium in amorphous form comprising extracting Atorvastatin hemi-calcium from a 2-methyl tetrahydrofuran solution followed by treating the Atorvastatin hemi-calcium with an anti-solvent to isolate amorphous Atorvastatin calcium.

8. The process of claim 1, wherein the anti-solvent is selected from cyclohexane, n-hexane, n-heptane, methyl-t-butyl ether or a mixture thereof.

9. A process for the preparation of [R—(R*,R*)]-2-(4-fluorophenyl)-β,δ-dioxane-5-(1-methylethyl)-3-phenyl-4-[(phenylamino)carbonyl]-1H-pyrrole-1-heptanoic acid tert-butyl ester, compound of formula III, 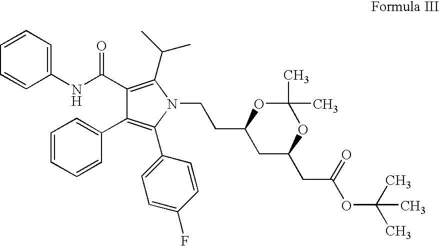
comprising:
a) reacting (4R-6R)-6-aminoethyl-2,2-dimethyl-1,3-dioxane-4-acetic acid tert-butyl ester (compound of formula IV) 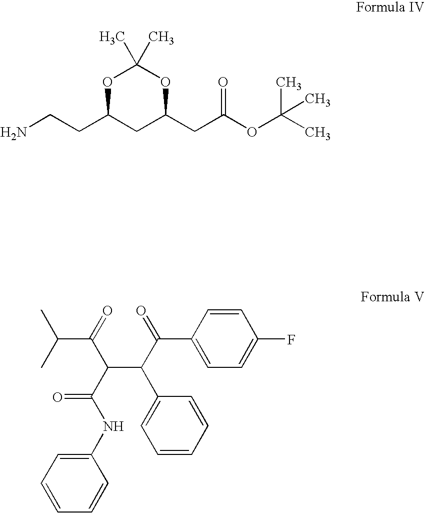
and 2-[2-(4-Fluoro-phenyl)-2-oxo-1-phenyl-ethyl]-4-methyl-3-oxo-pentanoic acid phenylamide (compound of formula V) in the presence of an acid catalyst and 2-methyl THF,
b) removing water azeotropically followed by distilling solvent from the reaction mixture formed in step (a), and
c) treating the product of step (b) with a C₁ to C₄ alcohol, aqueous alcohol or a mixture thereof.

10. The process of claim 9 wherein the acid catalyst in step (a) is selected from any of acetic acid, butyric acid, pivalic acid, benzoic acid, and trichloroacetic acid.

11. The process of claim 10 wherein the acid catalyst is pivalic acid.

12. The process of claim 9, wherein the C₁ to C₄ alcohol in step (c) is methanol, ethanol, or isopropanol.

13. The process of claim 9, wherein the C₁ to C₄ alcohol is an isopropyl-alcohol.

14. Atorvastatin hemi-calcium prepared by the process of claim 1.

15. The process of claim 7, wherein the anti-solvent is selected from cyclohexane, n-hexane, n-heptane, methyl-t-butyl ether or a mixture thereof.

16. Atorvastatin hemi-calcium prepared by the process of claim 7.

17. Atorvastatin hemi-calcium prepared by the process of claim 9.
